(19) United States
(12) Patent Application Publication
(10) Pub. No.: US 2016/0220235 A1
(43) Pub. Date: Aug. 4, 2016

(54) VASCULAR CLOSURE APPARATUS AND RELATED METHOD

(71) Applicant: ACCESS CLOSURE, INC., SANTA CLARA, CA (US)

(72) Inventor: ALI HASSAN ALMEDHYCHY, PALO ALTO, CA (US)

(73) Assignee: ACCESS CLOSURE, INC., SANTA CLARA, CA (US)

(21) Appl. No.: 15/082,745

(22) Filed: Mar. 28, 2016

Related U.S. Application Data
(63) Continuation-in-part of application No. 14/994,496, filed on Jan. 13, 2016.
Application No. 14/994,496 is a continuation of application No. 14/179,767, filed on Feb. 13, 2014, now abandoned.
(60) Provisional application No. 61/930,624, filed on Jan. 23, 2014.
(60) Provisional application No. 61/801,694, filed on Mar. 15, 2013.
(60) Provisional application No. 61/764,749, filed on Feb. 14, 2013.

(51) Int. Cl. A61B 17/00

(52) U.S. Cl. CPC A61B 17/0057; A61B 17/00491; A61B 2017/00659; A61B 2017/0065; A61B 2017/00575

(57) ABSTRACT

A vascular closure device can include both a mechanical component and a biological component for sealing an arteriotomy. The mechanical component reduces the size or closes the arteriotomy. The biological component covers and fills any spaces or cracks present after the arteriotomy has been mechanically reduced in size. An exemplary embodiment includes a suture or clip to approximate the edges of the arteriotomy, and a smooth rounded plug is advanced along the suture towards the closed arteriotomy. An applicator device can be used to deliver the plug and, in particular, the device can include a guidewire that is offset from a central axis along a side of the applicator. In situ, the plug transforms to a flowable or gel state and fills and covers any cracks and spaces along the closed arteriotomy.

### CROSS-REFERENCE TO RELATED APPLICATIONS

This application is a continuation-in-part of U.S. patent application Ser. No. 14/994,496, filed Jan. 13, 2016, which is a continuation of U.S. patent application Ser. No. 14/179,767, filed on Feb. 13, 2014, which claims priority to the following U.S. provisional patent application No. 61/764,749, filed Feb. 14, 2013; 61/801,694, filed Mar. 15, 2013; and 61/930,624, filed Jan. 23, 2014, the entirety of all of which are hereby incorporated by reference herein.

### BACKGROUND

The field pertains to medical devices, and more particularly, medical devices and methods designed for percutaneous vascular access closure.

Wounds such as arteriotomies can arise in the blood vessel from various medical procedures, especially for blood vessels acting as sites for catheter insertion during diagnostic and/or interventional catheterization.

Although such wounds may be fixed by application of pressure by hand, a number of vascular closure devices have been developed to address the need to close such wounds in a safe and more efficient manner.

Various closure devices tend to rely on either purely mechanical or purely biological means to close the wound.

US Patent Publication No. 2008/0249545 to Shikhman et al., for example, describes a mechanical based device. The Shikhman publication describes a percutaneous surgical device, which comprises a combination wound suturing and crimping and cutting device. In one exemplary embodiment a crimping and cutting device portion nests within a suturing device portion. The combined device may locate a vessel wound and pass suture through the vessel walls surrounding the wound. Then, the crimping and cutting portion may detach, the suturing portion may be removed, and the crimping and cutting portion may be located to the wound site to apply a fastener (e.g., a ferrule). See also Patent Publication Nos. 2010/0069930; 2004/0097968; 2003/0216755; 2003/0078601 also to Shikhman et al.

U.S. Pat. No. 7,060,078 to Hathaway et al. describes another mechanical based device. The '078 patent describes a device having two components: a needle advancing apparatus slidable longitudinally along a catheter to advance needles into a tissue membrane, such as a blood vessel wall, around an opening in the membrane; and, a suture retrieval assembly insertable through the catheter beyond a distal side of the tissue membrane. The needle advancing apparatus advances suture through the tissue wall. The suture retrieval assembly grabs the suture on the distal side of the tissue membrane for extraction thereof through the opening in the tissue membrane. A method for suturing a membrane beneath the patient's skin is also disclosed.

Such mechanical approaches tend to require precise positioning within the tissue tract, typically provide point (instead of a continuum of tissue purchase) support, and lead to permanent foreign-body implants that interfere with subsequent catheterization at the same vascular site. Additionally, a purely mechanical support of the wound could lead to implanting substantially non-absorbable foreign material that provides only point-support to the wound lips.

Various biological approaches to vascular closure are described in U.S. Pat. Nos. 5,108,421; 5,601,602 each to Fowler. In the '421 patent, a device and method of closing an incision or puncture in a patient is disclosed. The method includes inserting a vessel plug into the incision or puncture until the distal end of the vessel plug is adjacent to the outer lumen of the blood vessel. The vessel plug is positioned so that it does not obstruct the flow of fluid through the blood vessel or target organ. The precise positioning of the vessel plug in the incision or puncture is accomplished through the use of a balloon catheter or a cylindrical insertion assembly having a proximal plunger member associated therewith. See also U.S. Pat. No. 7,331,979 to Khosravi. Purely biological implants tend to provide relatively weak-mechanical support (especially in large wounds). Additionally, purely biological wound coverage, while being bioabsorbable, does not necessarily guarantee instant securing of the wound lips.

U.S. Pat. Nos. 5,021,059; 5,222,974; and US Patent Publication No. 2001/0003158 each to Kensey et al. describe another biological closure approach. The '059 patent, for example, describes deploying a collagen plug to seal the closure. In order to block the collagen from entering the vessel, a footplate is installed on the interior of the blood vessel. The footplate is held in place with a suture. The approaches described in the '059 patent, however, do not physically approximate the lips of the arteriotomy. The '059 patent describes a system which undesirably relies on only the collagen to close the wound.

It is thus desirable for a vascular closure device that is conveniently installed, that is efficient for the physician to deploy, and that addresses the above mentioned shortcomings.

### SUMMARY

The description, objects and advantages will become apparent from the detailed description to follow, together with the accompanying drawings.

A vascular closure device includes both a mechanical component and a biological component for sealing an arteriotomy. The mechanical component reduces the size or closes the arteriotomy. The biological component covers and fills any spaces or cracks (fissures) present after the arteriotomy has been mechanically reduced in size.

In one embodiment the device includes a suture or clip to approximate the edges of the arteriotomy, and a smooth rounded plug is advanced along the suture towards the closed arteriotomy. In another embodiment a sealant plug is advanced along a guidewire extending from the closed arteriotomy. In another embodiment a sealant plug is advanced alongside a guidewire extending from the closed arteriotomy. In situ, the plug transforms to a flowable or gel state and fills and covers any cracks and spaces along the closed arteriotomy.

In embodiments, the plug transforms from a solid structure to a more flowable gel state, and reconfigures to the shape of the puncture. In embodiments, the sealant is a PEG which cross links in situ. The sealant covers and fills the micropores arising from the mechanically closed lips of the arteriotomy. In a sense, the sealant is cast or molded in situ to match the puncture, fissures, and micropores created between the suture strands in the closed arteriotomy. Examples of sealants which may swell, reconfigure, and/or cross link in situ include biodegradable gels as described, for example, in US Patent Publication No. 2012/0209323.

In embodiments, the suture defines a central axis, and the sealant is advanced along an axis offset from the central axis. The sealant is directed towards the microspaces in the closed arteriotomy rather than merely on top of the suture bundle or knot. In other embodiments, the guidewire defines a central axis and extends directly from the micro spaces formed by the closed arteriotomy, and the sealant is advanced along the central axis towards the micro spaces so as to avoid merely being placed on top of the suture knots or bundles. These embodiments are intended to include a sealant being advanced on-axis or off-axis (namely, offset from the central guide axis).

In still other embodiments, an applicator or closure device used to place a sealant can incorporate a guidewire for positioning, where the guidewire is located in a side channel of the device, such that the guidewire avoids passing through the sealant where the sealant is directed through the applicator along a central axis and the guidewire is at an axis offset from the central axis. Thus, the sealant can avoid having a central hole therethrough yet can still utilize the guidewire located in a side channel of the device for positioning within the tissue tract.

In another embodiment, a surgical method for closing an open arteriotomy in a blood vessel comprises mechanically approximating a first lip of the arteriotomy to a second lip of the arteriotomy thereby forming a closed arteriotomy. The method further comprises covering the closed arteriotomy with a sealant.

In another embodiment a hybrid vascular access closure method and system provides both adequate mechanical support to the wound lips, and comprehensive sealing/coverage of the wound.

In another embodiment a method and system for vascular access closure minimizes the size of foreign-materials left in the tissue tract, without jeopardizing secure wound closure.

In another embodiment a system for large-bore vascular access closure is provided. In embodiments, a dilator is effective to enlarge the opening to a diameter ranging from 2 mm to 10 mm, and preferably, to at least 6 mm. In other embodiments, a smaller diameter dilator may be used to enlarge the opening.

In another embodiment a device and method include a biological space filler combined with minimum mechanical support to provide vascular closure.

In another embodiment a mechanical structure is deployed pre-procedure, thus providing assurance for the interventionalist during subsequent steps.

In another embodiment the mechanical component can provide a path (or reference point) for subsequent insertion of biological composition.

In another embodiment the mechanical component or biological component or both components include a radiopaque material.

In embodiments, a method and system comprise an additional feature for securing (e.g., cinching, tightening) the mechanical component, whereby the feature also serves as the biological component.

In embodiments, a sealant plug comprises a cable or zip tie opening. Free suture limbs extending from the lips of the arteriotomy are drawn through the tie opening, thereby approximating the tissue lips. After cinching, and the plug has been advanced through the tissue tract and urged against the exterior wall of the closed arteriotomy, the plug transforms from a first relatively firm state to a second gel-like (or flowable) state. The sealant flows across, covers, and fluidly seals the arteriotomy. The sealant also serves to mechanically secure (e.g., bond, weld, fix) the suture limbs. The tissue lips are held in a closed position.

### BRIEF DESCRIPTION OF THE DRAWINGS

FIGS. 1a-1o illustrate various steps of a medical procedures using a multimodality approach to vascular closure.

FIGS. 2a-2c show various mechanical-based techniques for vascular closure.

FIGS. 3a-3b show a balloon mediated tissue reorientation approach to reduce in size an arteriotomy.

FIG. 4 shows a biological-based component covering an arteriotomy.

FIGS. 5a-5b show a combination of mechanical and biological securing mechanisms for arteriotomy closure.

FIGS. 6a-6d show an arteriotomy closure approach by serial deployment of mechanical components, followed by biological components.

FIG. 7 shows an arteriotomy closure using mechanical and biological-based approaches.

FIG. 8a shows a perspective view of a closure plug including a suture locking channel.

FIGS. 8b and 8c are cross section and top views, respectively, of the plug shown in FIG. 8a.

FIG. 8d is an illustration of the plug shown in FIGS. 8a-8c placed in a tract and sealing an arteriotomy.

FIG. 9a shows a perspective view of a closure clip comprising suture locking apertures.

FIGS. 9b and 9c are side and top views, respectively, of the clip shown in FIG. 9a.

FIG. 10 shows an exploded view of a plug assembly including a bioabsorbable component and a mechanical component.

FIG. 11 is an illustration of the plug assembly shown in FIG. 10 placed in a tract and sealing an arteriotomy.

FIG. 12a shows an arteriotomy closure device using a biological-based approach where a guidewire is positioned in a side channel of the device.

FIG. 12b shows a cross-sectional view of the device of FIG. 12a along the lines b-b.

FIG. 12c shows a perspective view of a closure plug incorporated in the device shown in FIG. 12a.

### DETAILED DESCRIPTION

Before the present invention is described in detail, it is to be understood that this invention is not limited to particular variations set forth herein as various changes or modifications may be made to the invention described and equivalents may be substituted without departing from the spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail).

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Last, it is to be appreciated that unless defined otherwise, all technical and scientific terms used herein have the same meaning as commonly understood by one of ordinary skill in the art to which this invention belongs.

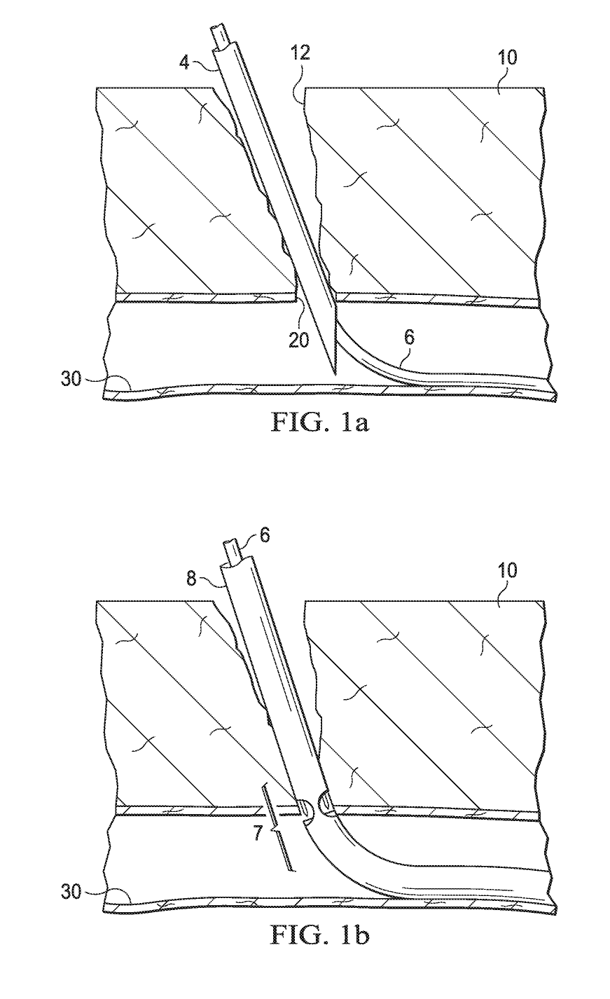

FIGS. 1a-1i illustrate vascular closure in accordance with one embodiment of the invention. Although FIGS. 1a-1i show percutaneous access to a blood vessel, the methods and devices are not intended to be so limited. The methods and devices may be utilized in procedures other than percutaneous procedures such as, for example, direct or open surgeries.

FIG. 1a shows an arteriotomy 20 in a blood vessel 30. A tissue tract 12 is shown leading from the exterior surface of the tissue 10 to the arteriotomy. An access needle 4 extends into the vessel. Additionally, a guidewire 6 is shown extending from the end of the needle and into the vessel 30.

After the vessel is accessed, needle 4 is removed leaving guidewire 6 in place.

Next, and with reference to FIG. 1b, a suturing device 8 may be fed over the guidewire 6 and into the vessel.

Alternatively, a sheath may be provided through which a suturing device is delivered to the vessel. See for example U.S. Pat. No. 8,197,510 to Nobles.

The suturing device 8 shown in FIG. 1b includes a tissue engaging section 7. The tissue engaging section is positioned across the lips of the arteriotomy. Suture is placed (or passed) through the lips of the arteriotomy using suturing device 8 and the suturing device is then removed. Typically, one or more inner needle members are deployed and capture suture ends. The suture ends may comprise features to enable capture such as ferrules or bulbs, loops, knots, and or hooks, etc. Indeed, a wide range of suturing devices may be used to pass the sutures. Exemplary suturing devices or means for placing sutures are described in U.S. Pat. No. 5,431,666 to Sauer et al., U.S. Pat. No. 6,641,592 to Sauer et al.; U.S. Pat. No. 5,304,184 to Hathaway; and U.S. Pat. No. 8,197,510 to Nobles.

FIG. 1c shows the suture 9 placed in the lips of the arteriotomy. Guidewire 6 is shown remaining in place for carrying out desired interventional or diagnostic procedures such as, for example, carrying out angioplasty, placement of a drug eluting stent, transvascular aortic valve implant, etc. Typically, in such procedures, the arteriotomy may be dilated with a dilating instrument. A vascular sheath is introduced over the guidewire and into the enlarged arteriotomy. The dilator may be insertable or used in combination with the sheath.

The desired medical procedure is performed through the vascular sheath. The vascular sheath and guidewire are removed, leaving the previously placed sutures.

FIG. 1d shows the arteriotomy reduced in size by physical or mechanical structures. The wound lips 20′ are shown mechanically approximated. Suture legs 9a, 9b are drawn or pulled with a force T to mechanically close the lips.

FIG. 1e shows advancing a delivery catheter 11 over the suture 9. Delivery catheter is advanced until distal end is adjacent the mechanically closed arteriotomy 20′. A biological plug 13 is disposed at the distal end of the delivery catheter and available for ejection. A wide range of delivery catheters to eject plug sealants may be used in accordance with the invention. Examples of delivery catheters and or instrument means for deploying a sealant are described in U.S. Pat. No. 8,382,797 to Khosravi et al. and U.S. Pat. No. 5,601,602 to Fowler.

In embodiments, the sealant can be introduced over the suture, or alongside the suture. The invention is intended to include all variations except as where specifically recited in the appended claims.

FIG. 1F shows deploying or ejecting the plug 13 and in particular, the delivery catheter outer tube is retracted while an inner support member 15 holds the sealant 13 in place and towards the arteriotomy. Consequently, the plug 13 is ejected and subject to reacting with the physiological materials of the tract. Preferably, plug 13 absorbs or otherwise reacts with the environment to change shape and properties which better serve to close the arteriotomy. In embodiments, the sealant transforms from a first relatively firm state 13 to a second more gelatinous state 13′. Examples of plug materials are described herein.

FIG. 1g shows tamping the plug 13′ by advancing (A) support member 15. This optional step serves to urge plug 13′ against the outside surface of the arteriotomy lips 20′, causing the gelatinous plug 13′ to fill micro-spaces or gaps left unclosed by the mechanical bond, and generally fill a section of the tissue tract 12.

FIG. 1h shows withdrawing (W) the delivery catheter 11 from the tissue tract 12. A cutting instrument or means (e.g. scissors) 17 are shown for cutting the suture limbs 9a, 9b. Sutures are preferably cut below the surface of the tissue 10.

FIG. 1i shows the mechanically approximated wound lips 20′ combined with wound coverage using biological sealant or moiety 13′. The sealant 13′ is shown covering the reduced or approximated arteriotomy 20′.

FIGS. 1j-1o illustrate vascular closure in accordance with another embodiment of the invention.

FIG. 1j shows an arteriotomy 402 partially closed with sutures 412, 414. Although two sutures are shown, the number of sutures applied to close the arteriotomy may vary. Additional sutures or clips may be deployed for larger openings. FIG. 1j also shows a guidewire 420 extending from the arteriotomy, and provides access to the arteriotomy. In particular, the guidewire 420 is extending from the slit between the sutures 412, 414.

FIG. 1k shows advancing a delivery catheter 430 over the guidewire 420. The sealant delivery catheter is advanced over the guidewire, through the tissue tract, and to the closed arteriotomy. The sealant is guided by the guidewire directly into the space between the sutures. In a sense, this is an example of an ‘on-axis’ delivery of the sealant. In other embodiments, sealant may be delivered off axis or off set from the guide member in order to optimally cover or fill the micro gaps left by the mechanically closed arteriotomy.

FIG. 1l shows delivery catheter distal end (or tip) adjacent the mechanically closed arteriotomy. A biological plug is disposed at the distal end of the delivery catheter and available for ejection. In embodiments, the sealant can be introduced over the guidewire, or alongside the member. The invention is intended to include all variations except as where specifically recited in the appended claims.

FIG. 1m shows ejecting the plug 440 and in particular, the delivery catheter outer tube is retracted while an inner support member holds the sealant in place and towards the arteriotomy. Outer sleeve containing sealant is retracted to reveal the sealant.

Consequently, the plug is deployed or ejected and subject to reacting with the physiological materials of the tract. Preferably, plug absorbs or otherwise reacts with the environment to change shape and properties which better serve to close the arteriotomy. In embodiments, the sealant transforms from a first relatively firm state to a second more gelatinous state. Examples of plug materials are described herein.

FIG. 1n shows tamping the plug 440 by advancing a support member 442 (e.g., a compression tube is advanced to compress sealant against artery wall). This optional step serves to urge plug against the outside surface of the arteriotomy lips, causing the gelatinous plug to fill micro-spaces or gaps left unclosed by the mechanical bond, and generally fill a section of the tissue tract.

FIG. 1o shows the guidewire and catheter removed from the tissue tract. The wound lips are both mechanically approximated and covered with the biological sealant 440 or moiety.

In embodiments the sealant additionally welds the suture limbs 9a, 9b together. In embodiments the suture and or plug are biodegradable and are fully absorbed over a time period. Preferably the materials are selected such that the time period is less than 90 days.

In embodiments, in situ, the sealant expands and migrates or flows into small spaces, cracks and micro-openings left remaining after the wound lips have been mechanically approximated.

In embodiments, the biological components can be polymers, proteins, other molecules, or a conjugation/combination of types of these components, individually or collectively designed to provide continuous wound coverage. Non limiting examples of biological sealants include biodegradable gels such as PEG, and collagens. The sealant may have a first plug shape, and transform to a flowable expandable gel, for example. Non limiting examples of biological sealants are described in U.S. Pat. Nos. 6,152,943; 6,165,201; 8,348,971; and 7,790,192 and US Patent Publication 2012/0209323 to Uchida et al.

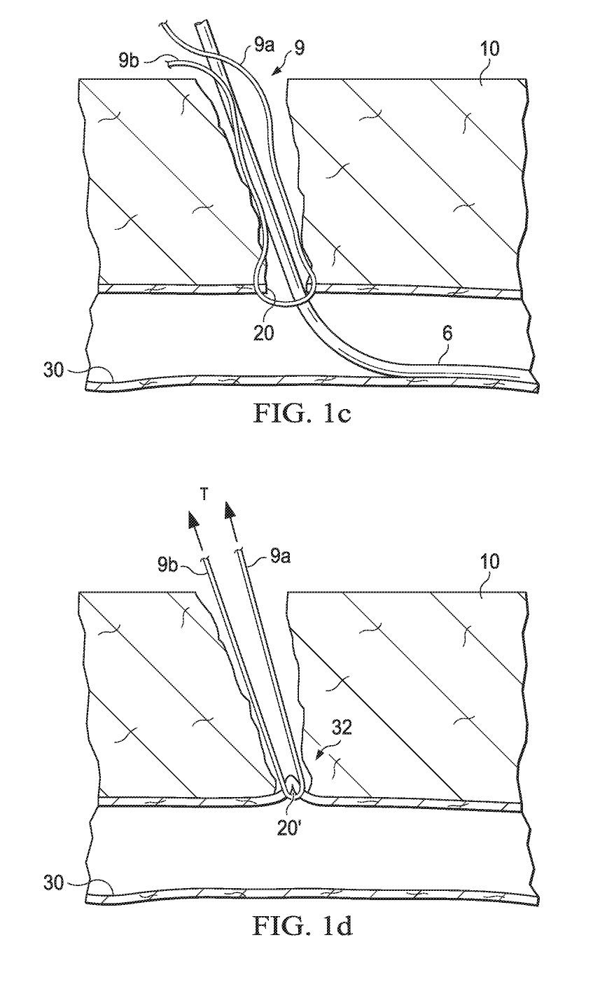

FIGS. 2a-2c show implants providing mechanical support (e.g., a mechanical component). Supports or links can include but are not limited to: sutures (reference numeral 50 of FIG. 2a), clips, staples (reference numeral 52 of FIG. 2b), hooks, scaffolds, disks, balloons (reference numeral 54 of FIG. 2c), and any other shapes designed to provide temporary or permanent wound support.

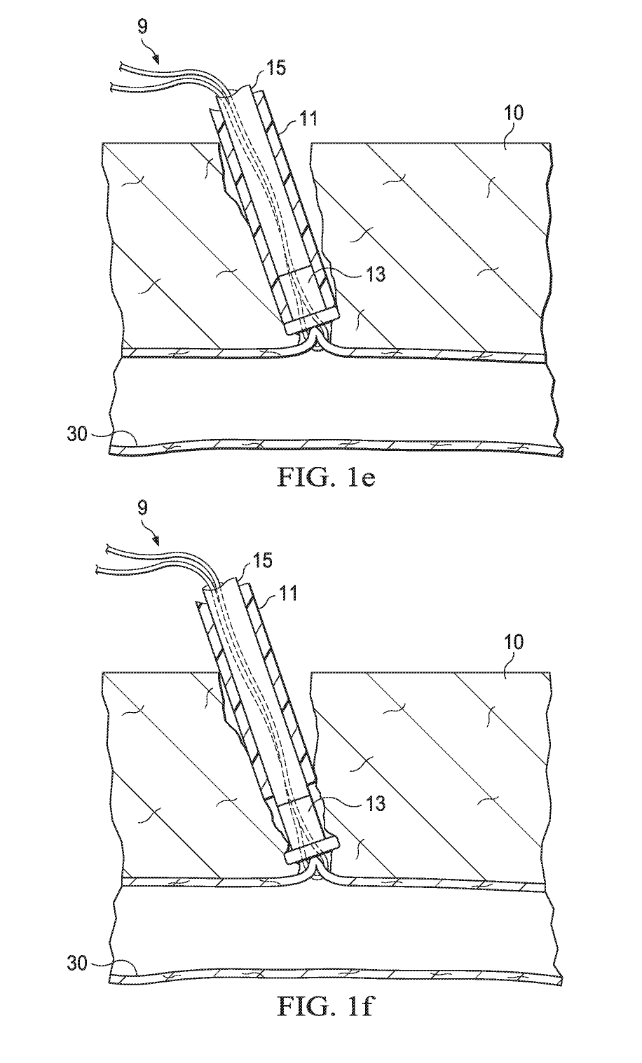

FIGS. 3a-3b shows tissue reorientation from an open wound 60, to a mechanically sealed closure 62, respectively. The tissue reorientation is desirably manipulated with a mechanical means or component. Exemplary mechanical functions include but are not limited to transiently pulling/grasping/clamping/reorienting tissue so as to create favorable conditions for a biologic to provide wound coverage thereupon the closure 62. In FIG. 3b, lips 64a, 64b of the wound 62 are oriented using member 66 (e.g., a balloon catheter). Balloon catheter includes a balloon 67 shown by hidden lines in FIG. 3b.

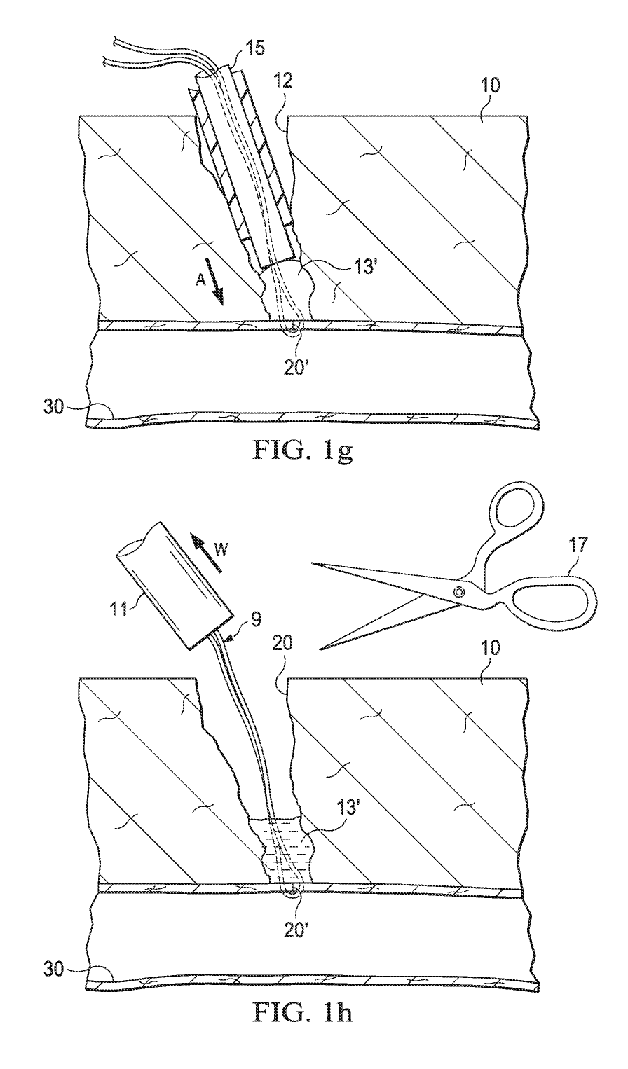

FIG. 4 shows wound coverage including the application of a biological sealant or moiety 110 to the wound. The sealant 110 acts as a space filler to seal the open wound of the vessel 112.

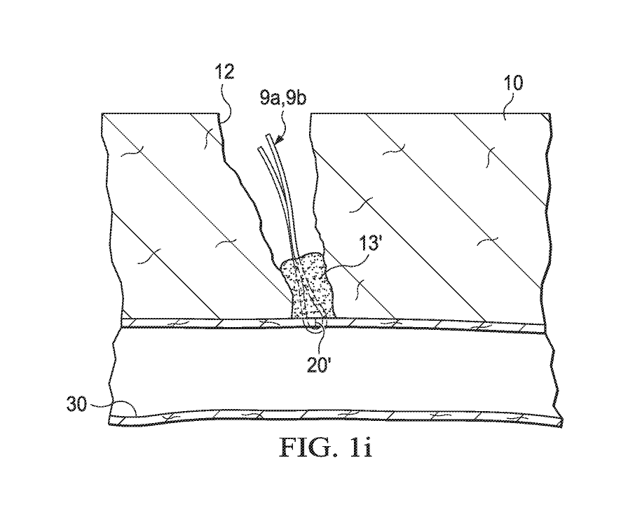

FIGS. 5a-5b show different views of closing a wound of blood vessel 120 using a combination of at least one mechanical securing mechanism 122 and one biological securing mechanism 124. As described herein, the biological components can be polymers, proteins, other molecules, or a conjugation/combination of types of these components, individually or collectively designed to provide continuous wound coverage.

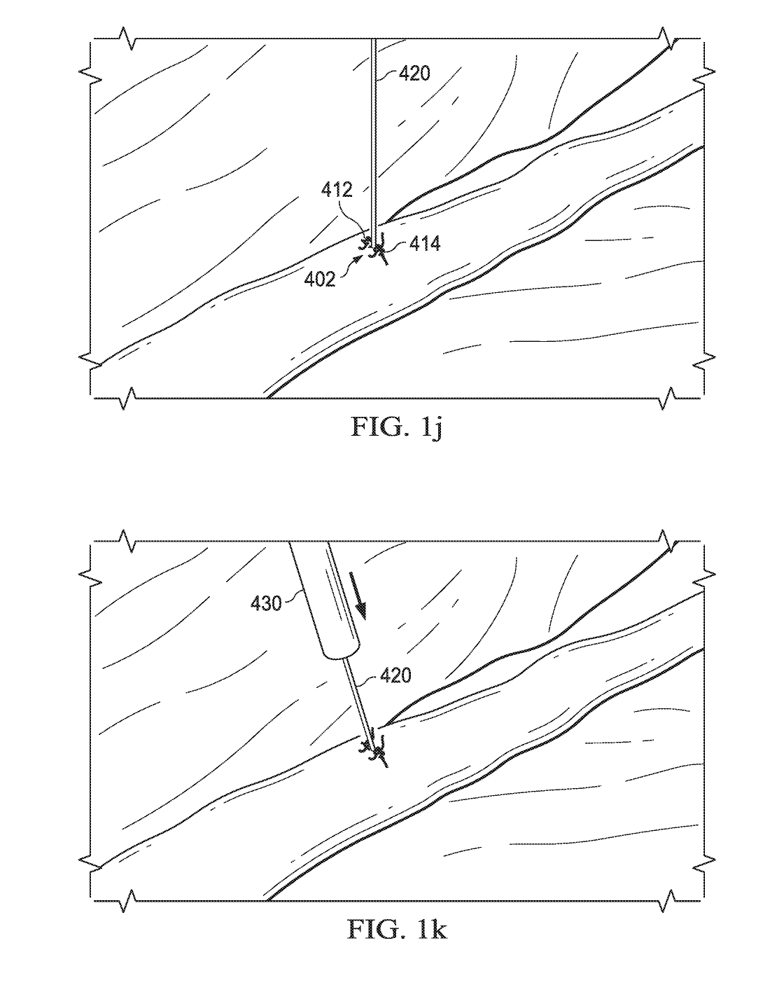

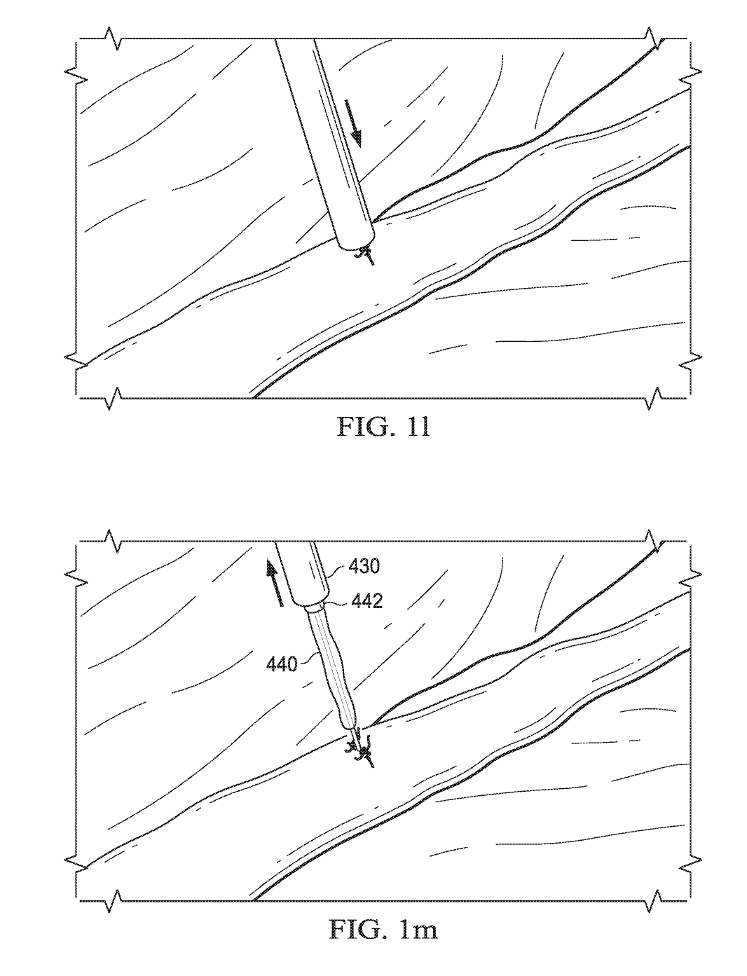

FIGS. 6a-6d, and 7 illustrate the administration of both biological and mechanical components to close a wound 200 of a blood vessel 202. FIG. 6a shows suture 204 installed across the open wound 200.

FIG. 6b shows the wound closed by virtue of sutures 204. Lips are shown approximated, forming a slit 208.

FIG. 6c illustrates advancing a biological sealant plug 206 along the sutures and towards the wound. Although not shown, a delivery catheter and/or support tube may advance the plug towards the site.

FIG. 6d illustrates the plug 206 covering the wound, and filling space on top and within portions of the wound. As described herein, the plug may be pushed along the sutures with a support tube or device. In embodiments, a method and system comprise an additional feature for securing (e.g., cinching, tightening) the mechanical component, whereby the feature also serves as the biological component.

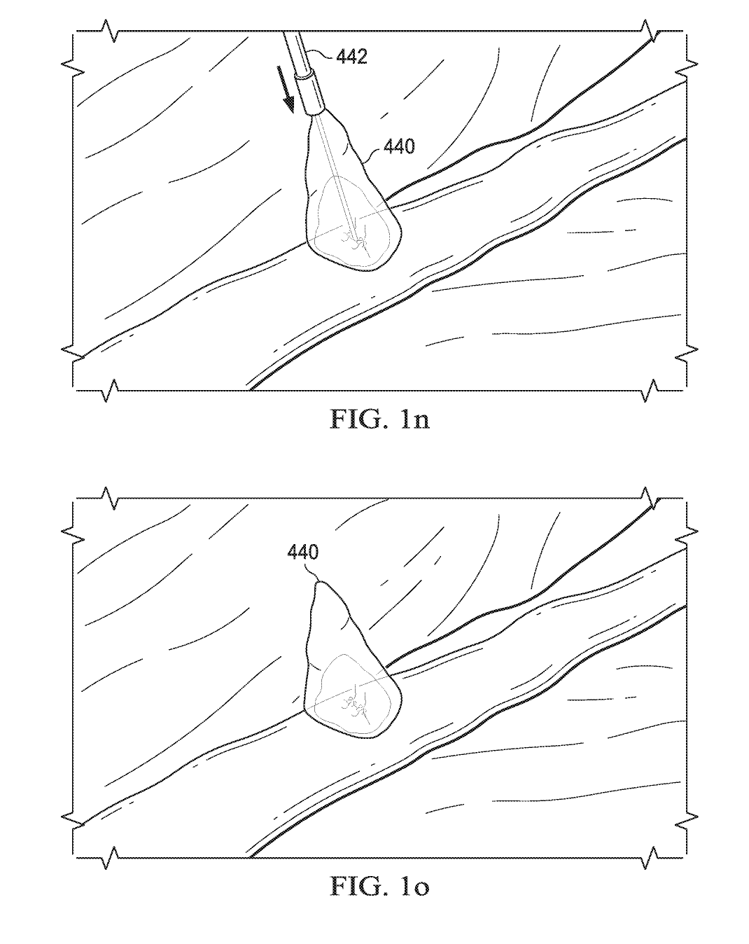

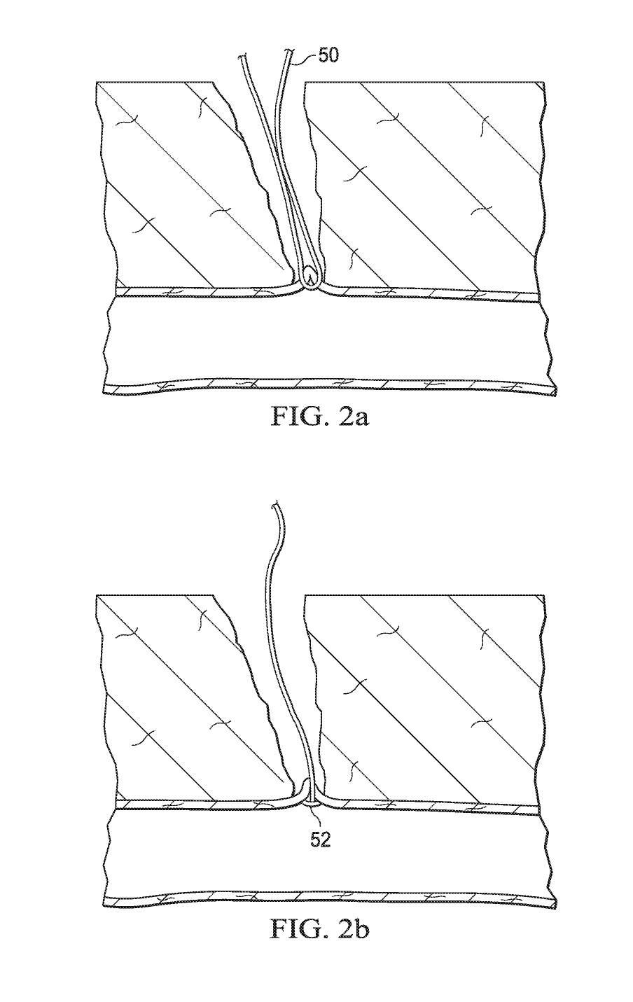

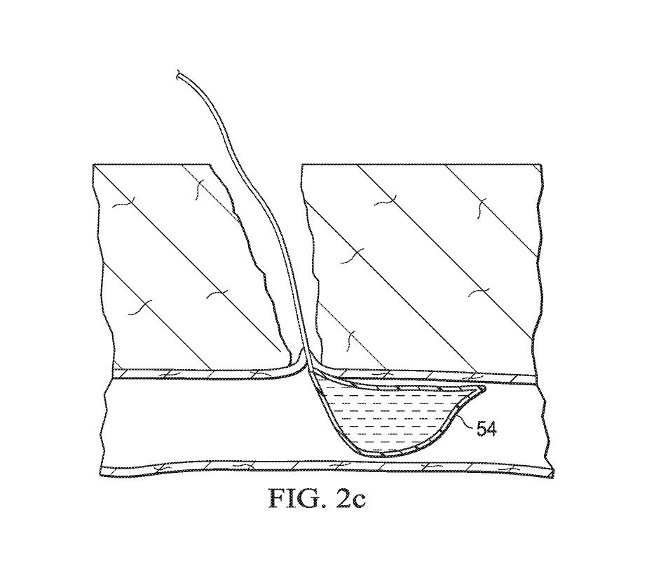

FIG. 8a shows a perspective view of a closure plug 300 including a suture locking channel 310. FIGS. 8b and 8c are cross section and top views, respectively, of the plug shown in FIG. 8a.

Suture channel 310 can have one or more clamping members 314. The clamps 314 are disposed at an angle to facilitate movement of the suture limbs (not shown) upwards (U). And to prohibit movement of the suture downwards (D).

FIG. 8c shows a slit 312 which is biased in a closed position. As the suture limbs (not shown) are drawn through the slit, the slit clamps onto the suture limbs.

With reference to FIG. 8D, in one embodiment, for example, a sealant plug 340 comprises a cable or zip tie opening. Free suture limbs 344 extending from the lips of the arteriotomy are drawn through the tie opening, thereby approximating the tissue lips. After cinching, and the plug has been advanced through the tissue tract and urged against the exterior wall of the closed arteriotomy, the plug transforms from a first relatively firm state to a second gel-like (or flowable) state. The sealant flows across, covers, and fluidly seals the arteriotomy. The sealant also serves to mechanically secure (e.g., bond) the suture limbs. Examples plug materials include those described herein. Consequently, the tissue lips are held in a closed position.

Optionally, suture legs may include enlarged sections, filled sections, bulbs, and other zip lock engagement features to allow engagement between the suture channel 310 and the suture legs 344.

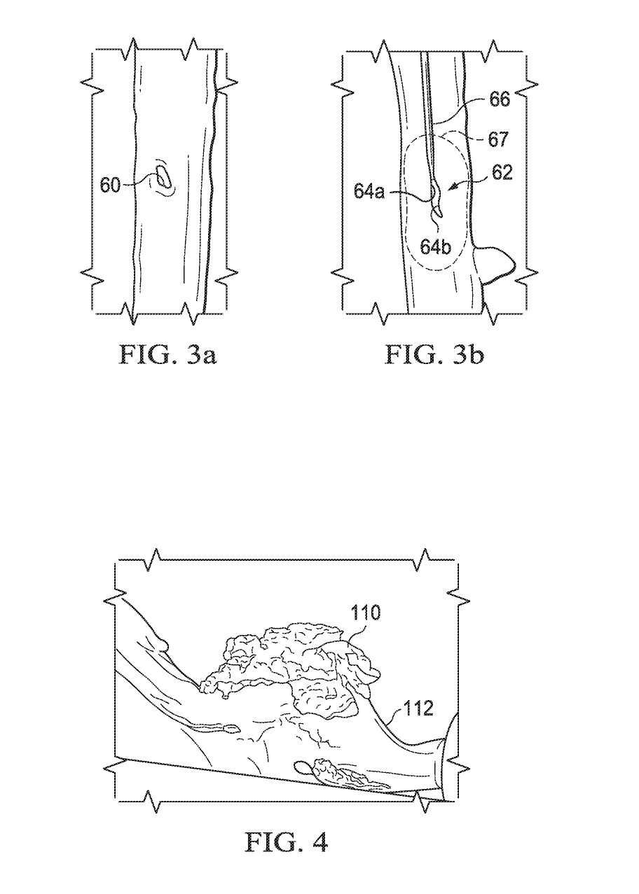

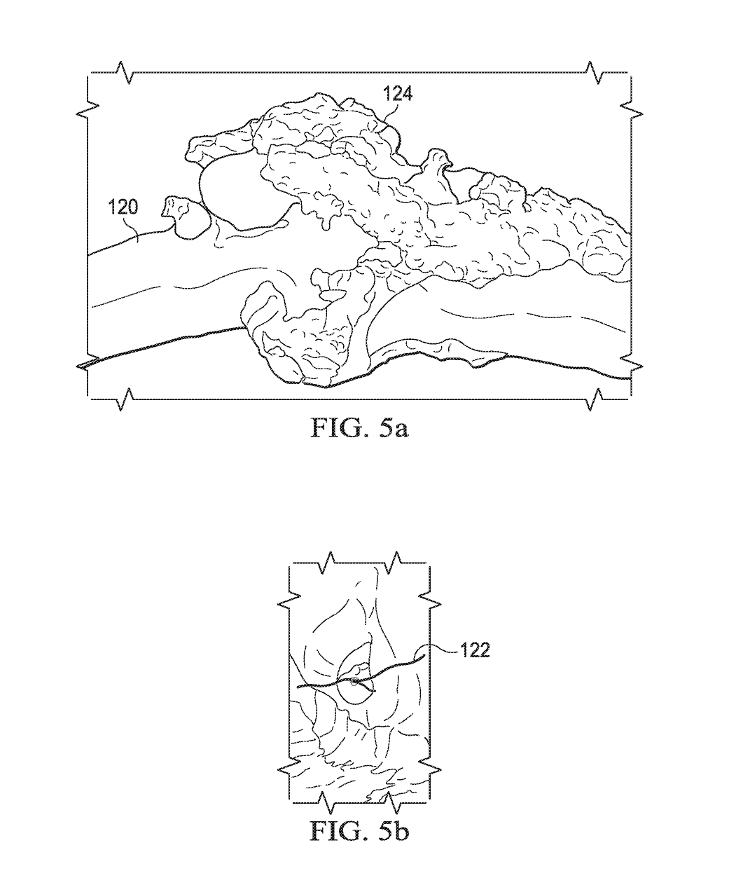

FIG. 9a shows a perspective view of a closure clip 320 comprising suture locking apertures 322a,b. FIGS. 9b and 9c are side and top views, respectively, of the clip shown in FIG. 9a. The clip structure is adapted to cinch down on the arteriotomy. The clip may be planar, and have cut-outs or apertures for receiving one or more sutures.

The apertures 322a, b are preferably spaced such that drawing the sutures through the apertures causes the lips to firmly shut. In one embodiment the clip has two circular shaped apertures and a space (G) separating the apertures ranging from 0.5 to 5 mm and preferably 0.5 to 1 mm.

The clip may have a thin, button or circular shape. Its thickness (t) may range from 1 to 5 mm, for example.

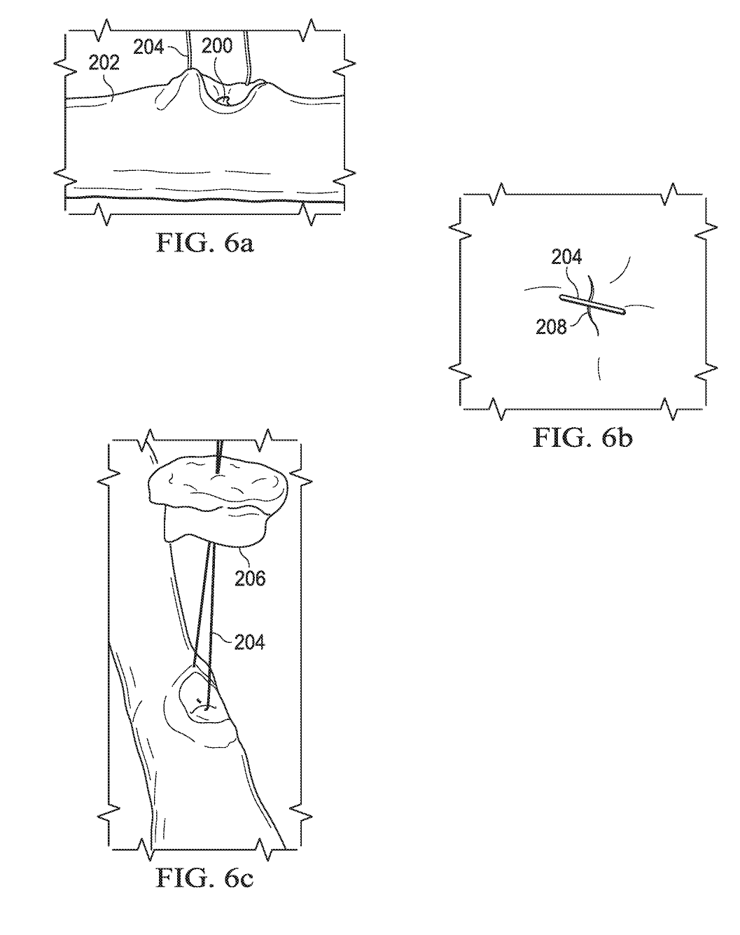

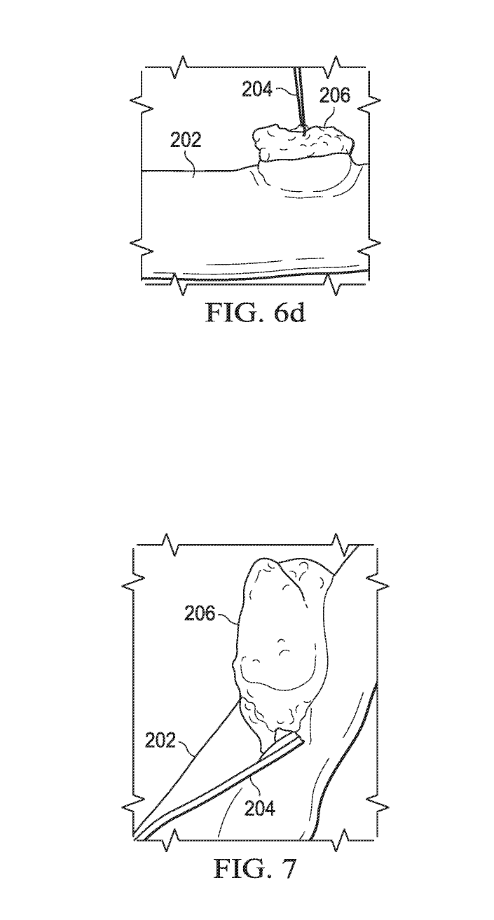

FIG. 10 shows an exploded view of a plug assembly including a bioaborbable component 340 and a mechanical component 342. FIG. 11 shows the assembly of FIG. 10 in an application. The biological assembly includes a sealant capsule 340 and a clip (e.g., a metallic clip) 342. The clip structure 342 is adapted to cinch the sutures 344 down on the arteriotomy. The clip may be planar, and have cut-outs or apertures for receiving one or more sutures. The clip may have a thin, button or circular shape.

In embodiments, the sealant capsule 340 is a reservoir or volume (e.g., a cylinder, or bullet shape) of sealant. A cylindrical shaped PEG is an exemplary sealant capsule.

The sealant capsule is advanced proximal to, distal to, or straddling the clip portion. The sealant capsule may be a component of the clip or reside within a cavity in the clip.

After cinching the sutures 344, and the biological assembly has been advanced through the tissue tract and urged against the exterior wall of the closed arteriotomy, the sealant capsule transforms from a first relatively firm state 340 to a second gel-like (or flowable) state 340′. The sealant flows across, covers, and fluidly seals the arteriotomy. The sealant also serves to mechanically secure (e.g., bond, weld, etc.) the suture limbs within the clip. Consequently, the tissue lips are held in a closed position.

FIG. 7 illustrates another view of a wound closed using the devices and method described herein, and in particular, the arteriotomy of blood vessel 202 is closed with a combination of mechanical component 204 and biological component 206. The mechanical member shown in FIG. 7 is a suture, which can be delivered using a suture delivery device. An example of a suture delivery or deployment device is described in, for example, U.S. Pat. No. 7,090,686 to Nobles et al., or as provided in other commercially available suturing devices.

The biological member shown in FIG. 7 is a formulation of hydro-gel different than that shown in FIG. 6d. The formulations of hydrogel can be already cross-linked, or designed to cross-link once deployed in the tissues as described herein.

The devices and components described herein may be deployed in various order. For example, FIGS. 6a-6d show serial deployment.

First, a mechanical component reduces the size of the wound as shown in FIGS. 6a-6b, then a sealant is advanced along the sutures 204 into a position covering the wound as shown in FIGS. 6c-6d.

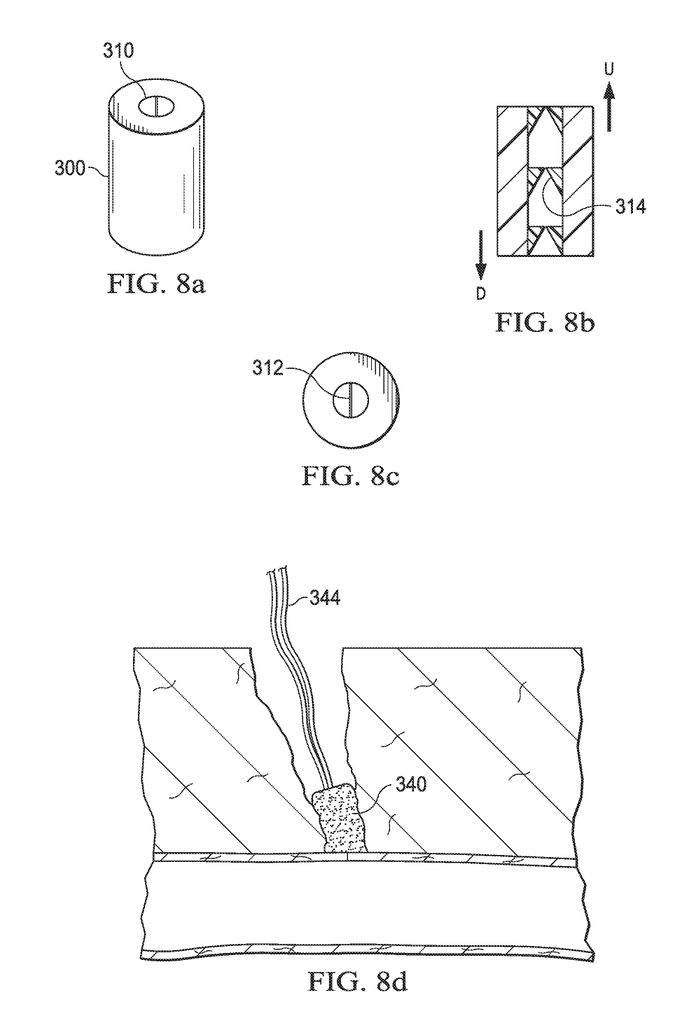

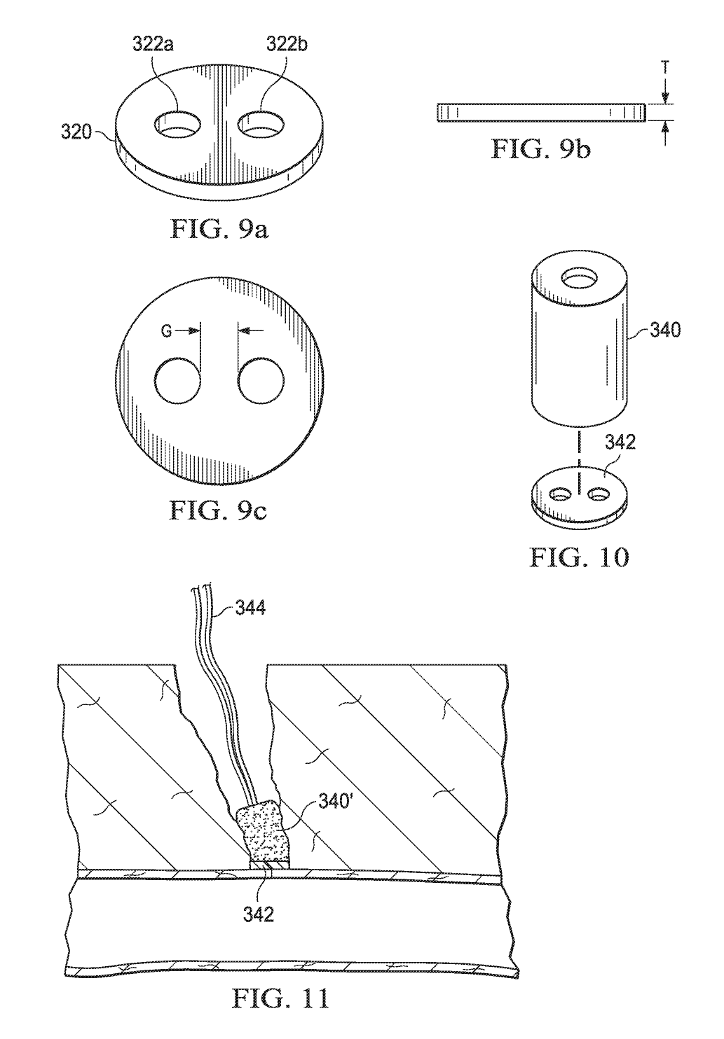

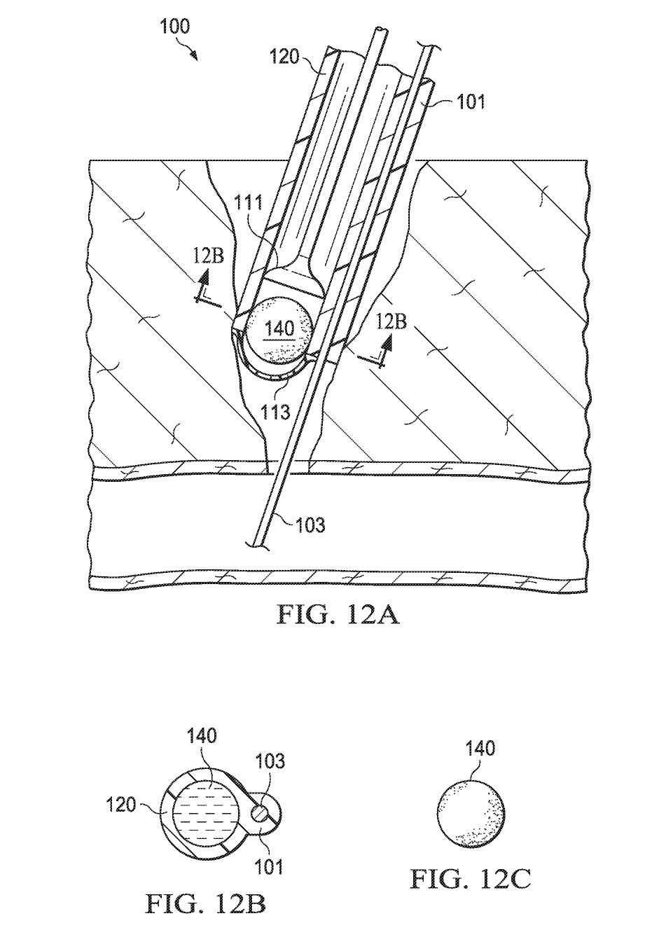

In another aspect, as shown in FIGS. 12a-12b, the applicator 100 can include a guidewire 103 that is located in a side channel or port 101 that avoids passing through the sealant 140, as seen in FIG. 12a, and is offset from the central axis. The guidewire channel 101 can be positioned adjacent to the sealant and/or the applicator without contacting the sealant. The side guidewire channel 101 can preserve the sealant 140 integrity and avoid adding a central hole in the sealant 140 by forcing the guidewire 103 to pass along the side of the applicator and, therefore, along the side of the sealant 140. Thus, the applicator 100 can be advanced and placed using the assistance of a guidewire 103, if necessary, but can still preserve the integrity of the sealant 140 by passing the guidewire 103 through a side port 101. The side port 101 can be provided along an external section of a shaft 120 of the applicator or it can be incorporated into the device as part of the shaft 120, i.e., internally, such that in either embodiment the side port 101 protrudes outward away from the diameter of the shaft 120, as seen in the cross-sectional view in FIG. 12b.

The guidewire channel 101 can be offset from the central axis, where the sealant is advanced along the central axis. This orientation allows the applicator to be advanced and positioned using the guidewire, without requiring additional guide members or sutures to act as a guide. Thus, the sealant 140 can be advanced after the mechanical closure procedure has taken place without requiring positioning assistance from the mechanical closure device. This is beneficial where a mechanical closure procedure has been performed and after the fact it is realized that there is still additional bleeding or leakage, which requires additional closure via providing the sealant. Alternatively, the sealant 140 can be placed as the only closure mechanism in the arteriotomy, without having any mechanical closure present. However, this alternative approach may require the aid of a temporary closure mechanism, such as a balloon or other expandable member to temporarily close the arteriotomy to prevent the sealant 140 from being ejected into the interior of the vessel; for the purposes herein a closed arteriotomy can refer to either a mechanically closed arteriotomy or a temporarily closed arteriotomy.

The sealant 140 can be any shape that is appropriate for placement of the sealant plug and, in one embodiment, the sealant can be a round, spherical sealant, as shown in FIG. 12c. As can be seen from the spherical sealant of FIG. 12c, the round sealant is completely smooth and does not need to contain any central holes or channels through the sealant. The rounded or spherical shape of the sealant can be beneficial for sealant placement. The rounded shape can allow the sealant to migrate toward the arteriotomy due to its shape and the characteristic of the sealant. Regardless of what shape sealant may be employed, the sealant of the embodiments in FIGS. 12a-12c can have a smooth, continuous interior and exterior, free of any holes or channels therethrough. The sealant 140 can be advanced along a central axis of the applicator while the guidewire is advanced along an axis offset from the central axis. Alternatively, a sealant with a central hole can also be used. The sealant, through amorphic expansion once exposed to the physiological fluids will expand and find the arteriotomy and its mechanically closed portion. Upon contact with physiological fluid present in the tissue tract, the sealant can flow onto and between the mechanically closed arteriotomy and fill at least one micro space created between suture strands in the mechanically closed arteriotomy.

The applicator 100, can be placed into the tissue tract toward the closed or, alternatively, still open arteriotomy. The applicator can be placed into the tissue tract after any previous mechanical closure techniques have been completed, e.g., after the arteriotomy has been mechanically closed. The applicator 100 containing the sealant 140 at its distal end can be advanced through the tissue tract over or alongside the guidewire 103 positioned in the adjacent side port 101. The applicator 100 can be advanced until the distal end is adjacent the mechanically closed arteriotomy. In one aspect, the distal tip 113 of the applicator 100 can have a rounded or curved tip such that the applicator can be advanced easier through the tissue tract and can avoid getting stuck on the interior wall of the tissue tract. Typically, when the puncture is made in tissue, the tissue walls can become uneven and difficult to pass through. A spherical distal tip 113 of the applicator can better propagate through the tissue tract between the skin and the arteriotomy.

Once the applicator 100 is in position adjacent the arteriotomy the sealant plug 140 can be ejected, as previously disclosed above. The applicator 100 can further include a pusher member 111, used to tamp the sealant by advancing the pusher member 111. This optional step can be used to urge the sealant 140 against the outside surface of the arteriotomy lips, e.g., the mechanically closed arteriotomy. Alternatively, the applicator 100 can also be placed into the tissue tract while the previous mechanical closure procedure is taking place or the device is still present in the tissue tract. Still alternatively, the applicator 100 can be placed into the tissue tract before or instead of a mechanical closure procedure utilizing some type of temporary closure mechanism prior to ejecting the sealant.

In embodiments, in situ, the sealant plug flows and expands into micro-openings to fluidly seal the wound in combination with the suture or other mechanical structures.

In embodiments, the mechanical and biological components may be deployed in parallel, or simultaneously. In embodiments, the components are deployed completely independent of each other.

In embodiments, the mechanical component can be transiently applied such as to provide initial wound support, and can be removed once the wound healing can be sustained with biological component alone.

In embodiments, the mechanical components or techniques for approximating the wound lips can include temporary or permanent implants, and/or techniques for tissue reorientation. An example of a temporary mechanical component is a biodegradable suture. An example of a permanent mechanical tissue approximation device is a metal clip or staple or non-absorbable suture.

In embodiments, the mechanical component or techniques for approximating the wound lips can include a temporary (PGLA, PLLA, hydrogel, etc) or permanent (Nitinol, stainless steel, platinum, titanium, etc) implant, and or techniques for securing mechanical component (suture, clip). The implant can comprise means for delivering biological component to the desired site.

In embodiments, the biological component can be: a smart moiety that selectively binds to domains in proximity of the wound; a moiety that infiltrates the wound and wound surrounding tissue such as to seal the wound; a composition of infiltrating and selectively binding moieties; and/or incorporate other materials/components that provide enhancement to the biological moiety such as reinforcement, visibility, expansion, etc. Examples of biological components include PEG, collagens, and hydrogels already cross linked to that cross link in situ.

In embodiments, the biological component can be solid or injectable, and it can comprise of a plurality of different biological forms. An example of a biological sealant is described in U.S. Pat. Nos. 6,152,943; 6,165,201, and 7,553,319.

In embodiments the biological component can be applied to wound proximity using an applicator, such as a catheter, or a part of a catheter system.

In embodiments, the biological component can operate in conjunction with a mechanical component, or uses a previously applied mechanical component as a reference during operation.

In embodiments, the biological component can operate as an adjunct, but is not dependent on mechanical component.

In embodiments an apparatus is configured to locate the wound level. For example, the applicator can be equipped with means to engage the wound, thereby providing reference for the wound level during operation, such means include balloons, expandable frames/shapes, structures temporarily/permanently attached to the wound etc. An example of a wound location configuration is described in U.S. Pat. No. 7,331,979.

In embodiments, the applicator can have a blunt tip, such as to stop at the wound level, and resists entering from the tissue tract into the vascular lumen.

In embodiments, the applicator can be equipped with means for preparing the tissue surrounding the wound. For example, the applicator can have an uneven, irregular, jagged, rough portion to dissect tissue away from the wound.

In embodiments, the applicator is adapted to provide a temporary seal of the wound for the duration of application of the moiety. For example, a balloon can be incorporated in the center of the applicator, that can be inflated such as to provide temporary seal of the vascular wound during operation.

In embodiments, the applicator is adapted to provide ports/channels for locally administering fluids/buffers/solutions aimed at catalyzing a chemical reaction within the biological moiety at wound level.

In embodiments, the applicator is adapted to provide a side port/channel for receiving a guidewire therethrough for directing and advancing the applicator without being delivered over a suture or other guide member.

In embodiments, the applicator is adapted to incorporate mechanisms to deploy and/or optimize mechanical component. For example, the applicator could be equipped with means to house an elongate object connected to pre-deployed mechanical components, thereby using the elongate object as a path to reach the mechanical component.

In embodiments, the applicator is equipped with an asymmetric/eccentric balloon that can be inflated intravascularly (beneath the wound); the asymmetric balloon can achieve desirable tissue reorientation transiently for subsequent steps.

Embodiments include any one or combination of the devices, methods, kit of apparatuses, systems, and implants as described herein.

Other modifications and variations can be made to the disclosed embodiments without departing from the subject invention.

We claim:

1. A surgical method for closing an open arteriotomy in a blood vessel, said method comprising:
mechanically approximating a first lip of the arteriotomy to a second lip of the arteriotomy thereby forming a closed arteriotomy;
advancing a sealant in an applicator toward the closed arteriotomy using a guidewire extending from a side of the applicator to aid in positioning the applicator and sealant adjacent the closed arteriotomy; and
ejecting the sealant from the applicator to cover the closed arteriotomy.

2. The method of claim 1, wherein the step of mechanically approximating is carried out with a suture.

3. The method of claim 1, wherein the sealant is spherical in shape.

4. The method of claim 1, wherein the sealant is free from any central hole.

5. The method of claim 1, wherein the applicator includes a side channel for receiving the guidewire therein, adjacent the sealant.

6. The method of claim 5, wherein the side channel is positioned on an exterior of the applicator.

7. The method of claim 5, wherein the side channel is positioned on an interior of the applicator.

8. The method of claim 1, further comprising inserting a sheath into the arteriotomy.

9. The method of claim 1, further comprising lubricating a tissue tract leading to the arteriotomy.

10. The method of claim 9, wherein the lubricating is carried out by sliding the sealant along the tissue tract towards the closed arteriotomy.

11. The method of claim 1 wherein the step of covering comprises applying a plug in a first configuration to the closed arteriotomy, and upon the plug being in situ, the plug taking a second configuration.

12. The method of claim 11, further comprising flowing the sealant into tissue cracks in the closed arteriotomy.

13. The method of claim 1, further comprising the step of tamping the sealant against the closed arteriotomy using a pusher member after ejecting the sealant.

14. The method of claim 1, further comprising adjusting the position of the sealant under fluoroscopy.

15. The method of claim 1, further comprising locating the exterior of the vessel wall.

16. The method of claim 1, wherein ejecting the sealant is performed without reinforcing the vessel wall from the inside.

17. The method of claim 1, wherein the applicator defines a central axis, and the sealant is advanced adjacent a guidewire offset from the central axis.

18. A surgical method for closing an open arteriotomy in a blood vessel, said method comprising:
placing suture through a first lip and a second lip of an arteriotomy thereby defining a first suture limb and a second suture limb extending from the first lip and the second lip respectively;
approximating the first lip to the second lip of the arteriotomy thereby closing the open arteriotomy into a closed arteriotomy, said closed arteriotomy comprising a slit having at least one micro space;
advancing a sealant towards the closed arteriotomy using an applicator device having a guidewire contained in a side channel adjacent the sealant but not in contact with the sealant;
ejecting the sealant once the applicator is positioned adjacent the closed arteriotomy; and
filling the micro space with the sealant.

19. The method of claim 18, wherein the sealant comprises a central axis, and the guidewire is along an axis offset from the central axis.

20. A surgical method for closing an arteriotomy in a blood vessel, said method comprising:
advancing a sealant in an applicator through a tissue tract toward an arteriotomy containing a mechanical closure;
positioning the applicator adjacent the mechanical closure using a guidewire extending from a side channel of the applicator; and
ejecting the sealant from the applicator into the tissue tract in close proximity of the arteriotomy, where upon contact with physiological fluid present in the tissue tract, the sealant expands and infiltrates onto and around the arteriotomy having the mechanical closure.

21. The surgical method of claim 18, wherein the applicator has a rounded distal tip.

22. The surgical method of claim 18, wherein the sealant is spherical in shape and does not contain a central channel therethrough.
